(19) United States
(12) Patent Application Publication
(10) Pub. No.: US 2004/0116516 A1
(43) Pub. Date: Jun. 17, 2004

(54) Pharmaceutical composition

(75) Inventors: Franz Birke, Ingelheim (DE); Hans Michael Jennewein, Wiesbaden (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/719,125

(22) Filed: Nov. 21, 2003

(30) Foreign Application Priority Data
Nov. 26, 2002 (EP) 02 026 223

(51) Int. Cl. A61K 31/325; A61K 31/277; A61K 31/155

(52) U.S. Cl. 514/478; 514/524; 514/634

(57) ABSTRACT

The invention relates to a pharmaceutical formulation comprising a LTB₄ antagonist having a hydroxy and a benzamidine group or a tautomer, a pharmaceutically acceptable salt, solvate, or physiologically functional derivative thereof, in particular a compound of formula (I)

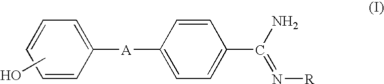

wherein R and A are as defined in the claims, or a tautomer, a pharmaceutically acceptable salt, solvate, or physiologically functional derivative thereof (1) and at least one cyclooxygenase-2 inhibitor, a combined cox1/2 inhibitor or a pharmaceutically acceptable salt, solvate, or physiologically functional derivative thereof (2), and a pharmaceutically acceptable carrier or excipient, and optionally one or more other therapeutic ingredients.

### BACKGROUND OF THE INVENTION

1. Technical Field

The invention relates to a pharmaceutical composition comprising certain oral available, LTB₄ antagonist, which contains a hydroxy and a benzamidine group, or a tautomer, a pharmaceutically acceptable salt, solvate, or physiologically functional derivative thereof (1) and at least one cyclooxygenase-2 inhibitor or a pharmaceutically acceptable salt, solvate, or physiologically functional derivative thereof (2), and a pharmaceutically acceptable carrier or excipient, and optionally one or more other therapeutic ingredients.

2. Background Information

The US patent U.S. Pat. No. 6,172,096 discloses a method of reducing recipient acute or chronic rejection of transplanted cells or organs, and for treatment of autoimmune diseases, hypersensitivity reactions of the acute or delayed type, allergic disorders, granulomas, meningitis, and septic shock by administering a cyclooxygenase-2 inhibitor and a leukotriene B₄ (LTB₄) receptor antagonist.

LTB₄ antagonists which contain a hydroxy and benzamidine group are compounds with pharmacologically valuable properties. Such LTB₄ antagonists may provide great therapeutic benefit, for example, in the treatment of arthritis, asthma, chronic obstructive lung diseases, psoriasis, ulcerative colitis, Alzheimer's disease, shock, reperfusion damage/ischaemia, cystic fibrosis, atherosclerosis and multiple sclerosis.

Such compounds are known e.g. from International Patent Applications WO 96/02497, WO 97/21670, WO 98/11062, WO 98/11119, WO 01/25186 and PCT/EP01/00262.

However, none of these prior art references indicates that the combination of a cyclooxygenase-2 inhibitor and a LTB₄ antagonists having a hydroxy and a benzamidine group will show a synergistic effect, in particular for the treatment of rheumatic arthtitis.

It has been demonstrated that arachidonic acid produced a dermal inflammation when applied topically (Carter et al, 5-Lipoxygenase inhibitory activity of zileuton. J Pharmacol Exp Ther 256, 929-937 (1990)). This inflammation is rich in neutrophils and consequently myeloperoxidase (MPO), a neutrophil marker enzyme, can be used as a quantitative index for cell infiltration. The mouse ear is especially suited to serve as a model of dermal inflammation induced by various agents like arachidonic acid which is known to be metabolized to several inflammatory mediators i.e. prostaglandines and LTB₄. Accordingly neither an LTB₄ antagonist nor an NSAID alone are supposed to fully counteract this kind of inflammation. Therefore this model seems to be useful to test the efficacy of a NSAID-LTB₄ antagonist combination.

It has now be found surprisingly that a pharmaceutical formulation comprising a cyclooxygenase-2 inhibitor and a LTB₄ antagonists having a hydroxy, preferably a phenolic hydroxy group and a benzamidine group shows a synergistic effect, in particular for the treatment of rheumatic arthtitis.

### BRIEF SUMMARY OF THE INVENTION

The invention relates to a pharmaceutical composition comprising a LTB₄ antagonists having a hydroxy and a benzamidine group, preferably a compound of formula (I)

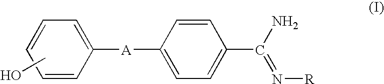

wherein

R represents a hydrogen atom or a group of formula —CO₂—R′, in which R′ represents a C₁₋₆ alkyl, an optionally substituted phenyl group or an optionally substituted benzyl group, wherein the optional substituents are selected from halogen atoms C₁₋₆ alkyl, C₁₋₆ alkoxy, cyano, nitro; C₁₋₆ haloalkyl and C₁₋₆ haloalkoxy groups, and

A is a group selected from the formulae (A1) and (A2):

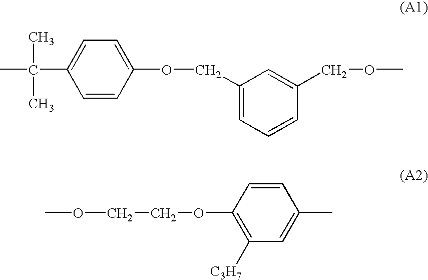

or a tautomer, a pharmaceutically acceptable salt, solvate, or physiologically functional derivative thereof (1), and

at least one cyclooxygenase-2 inhibitor or combined cox1/cox2 inhibitor or a pharmaceutically acceptable salt, solvate, or physiologically functional derivative thereof (2), and a pharmaceutically acceptable carrier or excipient, and optionally one or more other therapeutic ingredients.

R preferably represents H or —CO₂—C₂H₅.

Another aspect of the present invention is a method for the prevention or treatment of a disease or disorder selected from the group consisting of arthritis, including rheumatoid arthritis, spondyloarthropathies, gouty arthritis, osteoarthritis, systemic lupus erythematosus and juvenile arthritis, asthma, hay fever, atopic dermatitis, rhinitis, bronchitis, COPD and cystic fibrosis, psoriasis, sclerodermia, morbus bechterew, sarcoidosis, tumor metastasis, morbus crohn, colitis ulcerosa, IBD, multiple sclerosis, arteriosclerosis, arteritis, myocardial infarction, stroke, coronary heart disease which method comprises administration of effective amounts of a LTB₄ antagonist having a hydroxy and a benzamidine group, preferably a compound of formula (I) (1) and a cyclooxygenase-2 or combined cox1/cox2 inhibitor (2) to a patient in need thereof in a combined form, or separately or separately and sequentially wherein the sequential administration is close in time or remote in time.

Furthermore, the invention relates to the use of a LTB₄ antagonist having a hydroxy and a benzamidine group, preferably a compound of formula (I) (1) and a cyclooxygenase-2 inhibitor (2) in a combined form, or separately or separately and sequentially, wherein the sequential administration is close in time or remote in time, for the manufacture of a medicamentation for the prevention or treatment of disease or disorder selected from the group consisting of arthritis, including rheumatoid arthritis, spondyloarthropathies, gouty arthritis, osteoarthritis, systemic lupus erythematosus and juvenile arthritis, asthma, bronchitis, COPD and cystic fibrosis.

Finally, the invention relates to pharmaceutical kit comprising at least two separate unit dosage forms (A) and (B):

(A) one of which comprises a composition containing a LTB₄ antagonist having a hydroxy and a benzamidine group, preferably a compound of formula (I), a tautomer thereof or a pharmaceutically acceptable salt thereof (1), and optionally a pharmaceutically acceptable carrier;

(B) one of which comprises a composition containing one or more cyclooxygenase-2 inhibitors or combined cox1/2 inhibitors (2), and optionally a pharmaceutically acceptable carrier.

### DETAILED DESCRIPTION OF THE INVENTION

The term “LTB₄ antagonists which contain a hydroxy and benzamidine group” embraces compounds which selectively inhibit the leukotriene B₄ receptor and corresponding produgs thereof. They have preferably a “rod-like” structure of up to 5, preferably 3 or 4 phenylene moieties, which are connected to each other by linking groups selected from single bonds, straight chained or branched C₁₋₄-alkylenediyl, —O—C₁₋₄-alkylenediyl, C₁₋₄-alkylenediyl-O— and —O—C₁₋₄-alkylenediyl-O—. One of the said phenylene moieties, preferably a terminal one, carries a amidine group (—C(═NH)—NH₂), wherein the imino hydrogen atom may also be replaced by a capping group which enhances the bioavailability of the compound and is cleaved of under physiological conditions. Preferably one of the othe phenylene moieties, most preferably the other terminal one, carries a phenolic hydroxy group.

The term “capping group” preferably represents a group of formula —CO₂R′, wherein R′ has the meaning given hereinabove.

The term “C₁₋₆ alkyl” embraces straight chained and branched alkyl groups having 1 to 6 carbon atoms such as methyl, ethyl n-propyl, i-propyl, n-butyl, 2-butyl, n-pentyl and n-hexyl.

Preferred is a pharmaceutical composition, wherein the active principle essentially consists of a compound of formula (I), in particular formula (IA)

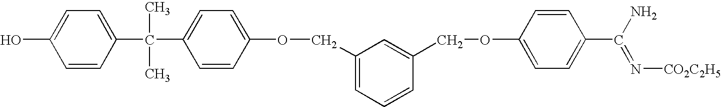

(1) and one cyclooxygenase-2 inhibitor or combined cox1/cox2 inhibitor.

The term “cyclooxygenase-2 inhibitor” embraces compounds which selectively inhibit cyclooxygenase-2 over cyclooxygenase-1, or which are combined cyclooxygenase-1 and cyclooxygenase-2 inhibitors.

Preferred are the cyclooxygenase-2 inhibitor or combined cox1/coxII inhibitor selected from the group consisting of celecoxib, Dupont Dup 697, etodolac, etoricoxib, flosulide, meloxicam, nimesulide, parecoxib, rofecoxib, Taisho NS-398 and valdecoxib, in particular meloxicam of formula

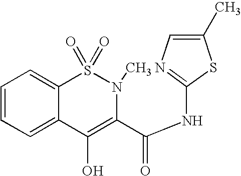

or a pharmaceutically acceptable salt thereof.

The phrase “combination therapy” (or “co-therapy”), in defining use of a cyclooxygenase-2 inhibitor agent and a leukotriene B₄ receptor antagonist agent, is intended to embrace administration of each agent in a sequential manner in a regimen that will provide beneficial effects of the drug combination. The phrase also is intended to embrace co-administration of these agents in a substantially simultaneous manner, such as in a single capsule having a fixed ratio of these active agents or in multiple, separate capsules for each agent.

The phrase “therapeutically-effective” is intended to qualify the amount of each agent for use in the combination therapy which will achieve the goal of improvement in severity and the frequency of disease incidence over treatment of each agent by itself, while avoiding adverse side effects typically associated with alternative therapies.

The active substance of formula I may be present in the formulation according to the invention in the form of a physiologically acceptable acid addition salt. By physiologically acceptable acid addition salts are meant, according to the invention, pharmaceutically acceptable salts which are selected from the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid. Mixtures of the above acids may also be used to prepare the salts. According to the invention, the preferred salts of formula I are selected from among the hydrochloride, hydrobromide, sulphate, phosphate, fumarate and methanesulphonate. The salts selected from among the hydrochloride, hydrobromide and fumarate are particularly preferred. The active substance may optionally be in the form of a hydrate. Preferably, according to the invention, the compound of formula I is added to the tablet in the form of the free base and in the anhydrous form.

The pharmaceutical formulation according to the present invention is as a rule suitable for oral, intravascular, intraperitoneal, subcutaneous, intramuscular or topical administration, in particular oral administration.

The present invention preferably relates to a tablet containing a compound of formula I and a cyclooxygenase-2 inhibitor or a combined cox1/cox2 inhibitor which contains at least one pharmacologically acceptable excipient, and optionally at least one wetting agent.

The term “wetting agent” as used hereinbefore and hereinafter denotes an excipient which lowers the surface tension of water or other liquids so that they can penetrate into the surfaces of the tablets according to the invention and soak through them, displacing the air, thereby wetting them. The substances used as wetting agents are usually interface-active surfactants. These surfactants are amphiphilic (bifunctional) compounds with at least one hydrophobic and one hydrophilic part of the molecule. The hydrophobic group is usually a hydrocarbon chain, if possible a straight chain, with eight to 22 carbon atoms. Particular surfactants may also have (dimethyl)-siloxane chains or perfluorinated hydrocarbon chains as the hydrophobic part of the molecule. The hydrophilic group is either a negatively or positively charged (hydratable) or a neutral polar head group. Of the surfactants, anionic surfactants, particularly the long-chain alkylsulphates, especially sodium laurylsulphate and alkylbenzenesulphonates are preferred.

Within the scope of the present invention carbohydrates such as lactose or mannose, particularly finely divided lactose or sugar alcohols such as mannitol, sorbitol or xylitol, particularly mannitol, are of particular importance as excipients. These excipients have proved particularly advantageous in the formulation according to the invention. In a preferred aspect, therefore, the present invention relates to a tablet containing at least one compound of formula I, which contains, in addition to the active substance and the wetting agent, lactose, particularly finely divided lactose, more preferably lactose monohydrate or mannitol as excipient.

The tablet according to the invention may also contain compounds capable of acting as binders.

The term “binder” used hereinbefore and hereinafter denotes excipients which are suitable for binding other components to one another. Preferred binders according to the invention are selected from among:

powdered cellulose, microcrystalline cellulose, sorbitol, starch, polyvinylpyrrolidone (povidone), copolymers of vinylpyrrolidone with other vinyl derivatives (copovidone), cellulose derivatives, particularly methylhydroxypropylcellulose, e.g. Methocel A 15 LV, and mixtures of these compounds. The preferred binders are powdered cellulose, particularly microcrystalline cellulose and/or copovidone. Most preferred is a mixture of microcrystalline cellulose and a copolymer of vinylpyrrolidone and vinyl acetate, namely copovidone VA 64, the ratio of vinylpyrrolidone and vinyl acetate being about 3:2 (m/m). As a rule the tablet according to the invention has a weight ratio of microcrystalline cellulose to copovidone VA 64 of 20:1 to 1:1, preferably 15:1 to 2:1, particularly about 10:1 to 3:1. Thanks to this particularly preferred binder combination of microcrystalline cellulose and copovidone, tablets are obtained having a high bioavailability of the compounds of formula I.

The tablet according to the invention may also contain disintegrants in addition to the above mentioned ingredients. Within the scope of the present invention these disintegrants may optionally also be known as breakdown agents. These are preferably selected, according to the invention, from among sodium starch glycolate, crosslinked polyvinylpyrrolidone (crospovidone), croscarmellose sodium salt (sodium salt of cellulose carboxymethyl ether, crosslinked), sodium-carboxymethylcellulose, dried maize starch and mixtures thereof. Within the scope of the present invention it is particularly preferred to use sodium starch glycolate, crospovidone and, preferably, the sodium salt of crospovidone or croscarmellose.

The tablet according to the invention may also contain flow agents or flow regulators and also lubricants, as additional ingredients. These include, within the scope of the present invention, for example, silicon dioxide, talc, stearic acid, sodium stearylfumarate, magnesium stearate and glycerol tribehenate. According to the invention magnesium stearate is preferably used.

In addition, the tablet according to the invention may contain one or more synthetic or natural, pharmaceutically acceptable dyes or colourings, preferably indigo carmine. If the abovementioned colourings are used the amount by weight thereof based on the total mass of the tablet according to the invention is 0.01 to 0.5 wt. %.

The active ingredients (1) and (2) are as a rule applied in a ratio, in which the resulting combination exhibits a synergistic effect. The term “synergistic effect” as used herein relates to an effect, which is higher than tone could expect from the additive effects of each single active ingredient.

Accordingly, the pharmaceutical formulation according to the present invention exhibits as a rule (1) and (2) in synergistically effective amounts of, preferably a weight ratio of (1) to (2), which ranges from 50:1 to 1:300, preferably from 8:1 to 1:80, in particular 1:1 to 1:40, most preferably 1:5 to 1:30.

The pharmaceutical formulation according to the present invention are preferably administered in a single application dose containing 1 to 10,000 milligrams, preferably 5 to 1000 mg of the combined active ingredients (1) and (2). Most preferred is a formulation comprising about 10 mg meloxicam and up to 300 mg of formula IA.

The Examples that follow serve to illustrate some formulations according to the invention. They are intended solely as possible procedures described by way of example, without restricting the invention to their content.

### EXAMPLE 1

| Ingredients | mg/tablet |
|---|---|
| (01) compound IA, jet-ground | 1,000 |
| (02) meloxicam | 10,000 |
| (03) microcrystalline cellulose | 15,000 |
| (04) mannitol | 52,250 |
| (05) croscarmellose sodium | 1,500 |
| (06) sodium laurylsulphate | 0,050 |
| (07) indigo carmine (11-14%) | 0,075 |
| (08) magnesium stearate | 1,125 |
| | 81,000 |

The direct compression comprises producing a premix of ingredients (01), (02), (06), (07) and some of (04) with an intensive mixer. The premix is screened and mixed with ingredients (03), (05) and the remainder of (04) in a gravity mixer. After the mixture has been screened again, ingredient (08) is added.

### EXAMPLE 2

2.1 Animals

Female albino mice (Han:NMRI) obtained from Interfauna and weighing about 20-25 g were used. The animals were provided with standardised pellet diet (Altromin 8013) and had tap water freely available. The animals were accommodated in a climatized room with a 12 hours light/dark cycle and kept in groups.

2.2 Chemical Substances

Compound of Formula IA:

Carbamic acid, [[4-[[3-[[4-[1-(4-hydroxyphenyl)-1-methylethyl]-phenoxy]methyl]phenyl]methoxy]phenyl]iminomethyl]-, ethyl ester was prepared as described in U.S. Pat. No. 6,417,382.

Meloxicam was provided by Boehringer Ingelheim Pharma KG

Arachidonic acid was purchased from Sigma (A9798) and dissolved 1:10 in acetone.

2.3 Study Design

The compound of formula IA and meloxicam were administered orally (0.2 ml/10 g bw) 30 min. before arachidonic acid challenge. Meloxicam was given twice: one dose 16 hours and the second dose 30 minutes before challenge. For every day there was a concurrent control. Then number of animals per group was 7. The study compounds were suspended in 1% methylcellulose (Tylose MH 300, Fluka, CH-9470 Buchs). The experiment was run in five groups. Details are given in Table 1. Every group contained one control, two doses of meloxicam, two doses of (IA), and one dose of the combination of the two compounds.

TABLE 1

| | | Dose | Dose | Dose | Dose | Dose |
|---|---|---|---|---|---|---|
| | | m/kg | m/kg | m/kg | m/kg | m/kg |
| | | p.o.) | p.o.) | p.o.) | p.o.) | p.o.) |
| Treatment | N | Group 1 | Group 2 | Group 3 | Group 4 | Group 5 |
| Control | 7 | — | — | — | — | — |
| (Tylose) | | | | | | |
| Meloxicam | 7 | 1 | 2 | 4 | 8 | 16 |
| Meloxicam | 7 | 2 | 4 | 8 | 16 | 32 |
| (IA) | 7 | 0.05 | 0.1 | 0.2 | 0.4 | 0.8 |
| (IA) | 7 | 0.1 | 0.2 | 0.4 | 0.8 | 1.6 |
| Meloxicam | 7 | 1 | 2 | 4 | 8 | 16 |
| plus (IA) | | 0.05 | 0.1 | 0.2 | 0.4 | 0.8 |

2.4 Experimental Procedure

Mice were lightly anesthetized by ether and 1 mg arachidonic acid (10 μl) was applied to each side of the left ear. The right ear remained untreated, acetone alone did not cause any late response. The animals were sacrificed by ether 6 hours later, and a biopsy (diameter 8 mm) was punched out from both ears to assess an increase of neutrophils in the left ear compared with the right ear. Tissue samples were homogenized in 1 ml 0.5% HTAB (Hexadecyl-trimethyl-ammonium-bromide; Sigma H-5882; solved in 0.05 M phosphate buffer, pH 6.0) using a tissue homogenizer (IKA-Ultraturrax T5; Janke & Kunkel, Staufen/Breisgau) at 30000 RPM for 15 seconds. After centrifugation (16000 G, 5 min) the supernatants were frozen until processing for myeloperoxidase (MPO). Determination in the supernatants for MPO, a neutrophil marker enzyme, served as a quantitative index for the neutrophil accumulation.

MPO was determined spectrophotometrically at 450 nm using a micro plate version of the method of Bradley (1982) and a micro plate reader (Vₘₐₓ; Molecular Devices, Palo Alto) suitable for kinetic measurements and expressed as mean optical density per minute.

2.5 Statistical Evaluation

For each individual the response myeloperoxidase activity (MPO) in optical density per minute (oD/min) was measured.

Based on these results the following statistical analyses were done:

Comparison of control groups

Comparison of Control vs. Treatments separately for each group

Comparison of high vs. low dose for both treatments separately for each group

Comparison of combined treatment vs. high doses separately for each group

Comparison of combined treatment vs. other treatments in group 5

For the comparisons the nonparametric statistical methods Kruskal-Wallis-One-Way-ANOVA and the Wilcoxon-Two-Sample-Test (2 sided) have been used. For the first and last comparisons also Bonferroni-Holm adjusted p-values have been calculated.

The calculations and statistical analysis were done with the NPAR1WAY procedure of the SAS software program (SAS Institute Inc., Cary, N.C.) version 8.2. (IA) and meloxicam both inhibited arachidonic acid induced ear inflammation in mice. Details of statistical analysis are shown in tables 2 to 4.

TABLE 2
Difference between treatment and control

| | Treatment | | p-value |
|---|---|---|---|
| Group | Compound/dose [mg/kg] | p-value | (adj) |
| 1 | Meloxicam/1 | 1.0000 | n.s. |
| | Meloxicam/2 | 0.2502 | n.s. |
| | (IA)/0.05 | 0.7983 | n.s. |
| | (IA)/0. | 0.7491 | n.s. |
| | Meloxicam/1 + (IA)/0.05 | 0.8983 | n.s. |
| 2 | Meloxicam/2 | 0.0152 | 0.0304 |
| | Meloxicam/4 | 0.1252 | 0.1252 |
| | (IA)/0.1 | 0.0060 | 0.0240 |
| | (IA)/0.2 | 0.0073 | 0.0219 |
| | Meloxicam/2 + (IA)/0.1 | 0.0022 | 0.0110 |
| 3 | Meloxicam/4 | 0.0736 | 0.0736 |
| | Meloxicam/8 | 0.0049 | 0.0147 |
| | (IA)/0.2 | 0.0073 | 0.0146 |
| | (IA)/0.4 | 0.0033 | 0.0165 |
| | Meloxicam/4 + (IA)/0.2 | 0.0033 | 0.0165 |
| 4 | Meloxicam/8 | 0.3067 | n.s. |
| | Meloxicam/16 | 0.7015 | n.s. |
| | (IA)/0.4 | 0.0553 | 0.1659 |
| | (IA)/0.8 | 0.0106 | 0.0424 |
| | Meloxicam/8 + (IA)/0.4 | 0.0022 | 0.0110 |
| 5 | Meloxicam/16 | 0.1599 | n.s. |
| | Meloxicam/32 | 0.2246 | n.s. |
| | (IA)/0.8 | 0.0049 | 0.0196 |
| | (IA)/1.6 | 0.0072 | 0.0216 |
| | Meloxicam/16 + (IA)/ | 0.0017 | 0.0085 |
| | 0.8 | | |

TABLE 3
Comparisons High/Low Treatment and High Treatment vs Combination

| Group | Low Dose [mg/kg] | High Dose [mg/kg] | p-value |
|---|---|---|---|
| 1 | Meloxicam/1 | Meloxicam/2 | 0.3067 |
| | (IA)/0.05 | (IA)/0.1 | 0.8983 |
| 2 | Meloxicam/2 | Meloxicam/4 | 0.7983 |
| | (IA)/0.1 | (IA)/0.2 | 0.7983 |
| 3 | Meloxicam/4 | Meloxicam/8 | 0.0474 |
| | (IA)/0.2 | (IA)/0.4 | 0.5229 |
| 4 | Meloxicam/8 | Meloxicam/16 | 0.7983 |
| | (IA)/0.4 | (IA)/0.8 | 0.9490 |
| 5 | Meloxicam/16 | Meloxicam/32 | 0.4320 |
| | (IA)/0.8 | (IA)/1.6 | 0.7012 |
| Group | High Dose [mg/kg] | Combination[mg/kg] | p-value |
| 1 | Meloxicam/2 | Meloxicam/1 + (IA)/0.05 | 0.1792 |
| | (IA)/0.1 | Meloxicam/1 + (IA)/0.05 | 0.8983 |
| 2 | Meloxicam/4 | Meloxicam/2 + (IA)/0.1 | 0.0033 |
| | (IA)/0.2 | Meloxicam/2 + (IA)/0.1 | 0.0348 |
| 3 | Meloxicam/8 | Meloxicam/4 + (IA)/0.2 | 1.0000 |
| | (IA)/0.4 | Meloxicam/4 + (IA)/0.2 | 0.3067 |
| 4 | Meloxicam/16 | Meloxicam/8 + (IA)/0.4 | 0.0033 |
| | (IA)/0.8 | Meloxicam/8 + (IA)/0.4 | 0.0215 |
| 5 | Meloxicam/32 | Meloxicam/16 + (IA)/0.8 | 0.0026 |
| | (IA)/1.6 | Meloxicam/16 + (IA)/0.8 | 0.0086 |

TABLE 4
Comparisons Combination vs Others for Group 5

| Comparison vs | | p-value |
|---|---|---|
| combined (Group5) | p-value | (adj) |
| Control | 0.0017 | 0.0085 |
| Meloxicam/16 | 0.0032 | 0.0096 |
| Meloxicam/32 | 0.0026 | 0.0104 |
| (IA)/0.8 | 0.0090 | 0.0090 |
| (IA)/1.6 | 0.0086 | 0.0172 |

The arachidonic acid induced mouse ear inflammation test is indicative for pathological processes where neutrophils are involved (see also introduction). It shed light especially on the chemoattractant part of the arachidonic acid cascade induced neutrophil activation. The results indicate that (IA) and Meloxicam are effective orally and consequently may target important parts of this inflammation.

With respect to the combination of both compounds, there are two criteria which must be fulfilled to prove a super-additive efficacy (potentiation).

i.) the maximal achievable effect of the combination must be bigger than the maximal achievable effect of the single compound.

ii.) the effect of the combination should be bigger than the effect which can be expected from the dose response curve of the single compounds

The first criterion is experimentally tested by using supra-maximum doses of the single compounds and compare these with the achievable maximal effect of the combination. This may be difficult when the compounds under investigation produce a 100% inhibition itself. Therefore the model of arachidonic acid induced mouse ear inflammation was chosen, because NSAIDs and LTB₄ antagonists demonstrate very shallow dose response curves and a 100% inhibitory effect is hardly possible. As shown in the present experiments of group5 the supra-maximum doses of meloxicam (16 and 32 mg/kg p.o.) and (IA) (0.8 and 1.6 mg/kg p.o.) achieved maximum inhibitory effects o 37% and 66% respectively, whereas the combination (meloxicam 16 mg/kg p.o., (IA) 0.8 mg/kg p.o.) achieved a maximum inhibition of 96%. This difference was statistically significant and consequently proves a super-additive effect according to criterion i.).

The second criterion can be tested by doubling the doses of the single compounds and compare the effect of the higher doses of the single compounds with the effect of the combination of the lower doses of the single compounds. In group 1 all doses were too low to achieve any effect and consequently the results of this experiment cannot be used either to accept or reject the hypothesis of potentiation. The same holds true for the experiments in group 3. Although the combination achieved the highest inhibition, the difference to the inhibition reached with the highest doses of the single compounds were not statistically significant, because the single compounds alone caused already high inhibition values. However the experiments performed in group 2, 4, and 5 clearly show that the combination was significantly more effective than the higher doses of the single compounds thus proving a super-additive efficacy according to criterion i.i.

It is concluded that the combination of the non steroidal anti inflammatory drug meloxicam with the LTB₄ antagonist (IA) strongly inhibits arachidonic acid induced mouse ear inflammation after oral administration in an super-additive way

1. A composition comprising a LTB₄ antagonist having a hydroxy and a benzamidine group, or a tautomer, a pharmaceutically acceptable salt or solvate thereof (1), and a cyclooxygenase-2 inhibitor or combined cox1/coxII inhibitor or a pharmaceutically acceptable salt or solvate thereof (2), and a pharmaceutically acceptable carrier or excipient.

2. The composition according to claim 1 wherein said LTB₄ antagonist is a compound of formula (I) 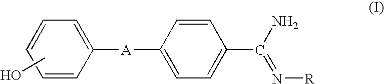
wherein
R represents a hydrogen atom or a group of formula —CO₂—R′, in which R′ represents a C₁₋₆ alkyl, an optionally substituted phenyl or an optionally substituted benzyl group, wherein the optional substituents are selected from halogen atoms C₁₋₆ alkyl, C₁₋₆ alkoxy, cyano, nitro; C₁₋₆ haloalkyl and C₁₋₆ haloalkoxy groups, and
A is a group selected from the formulae (A1) and (A2): 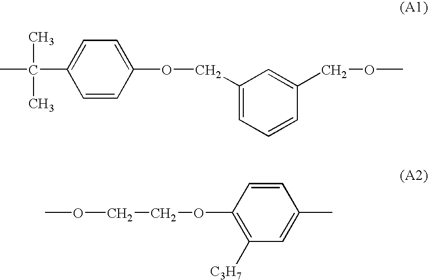
or a tautomer, a pharmaceutically acceptable salt or solvate thereof (1).

3. The composition according to claim 2 consisting essentially of the compound of formula (IA) 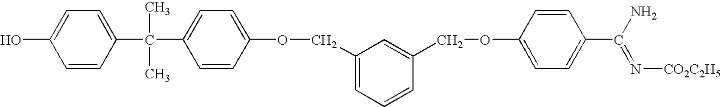
(1) and a cyclooxygenase-2 inhibitor or combined cox1/coxII inhibitor selected from the group consisting of celecoxib, Dupont Dup 697, etodolac, etoricoxib, flosulide, meloxicam, nimesulide, parecoxib, rofecoxib, Taisho NS-398 and valdecoxib or a pharmaceutically acceptable salt or solvate thereof (1), and a pharmaceutically acceptable carrier or excipient.

4. The composition according to claim 3 comprising the compound of formula (IA) (1) and meloxicam of formula 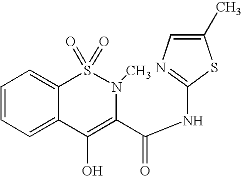
or a pharmaceutically acceptable salt thereof (2), and a pharmaceutically acceptable carrier or excipient.

5. The composition according to claim 1 which is in a form suitable for oral, intravascular, intraperitoneal, subcutaneous, intramuscular or topical administration.

6. The composition according to claim 1 wherein the weight ratio of (1) to (2) ranges from 50:1 to 1:300.

7. The composition according to claim 1 wherein a single application dose contains 1 to 10,000 milligrams of the combined active ingredients (1) and (2).

8. The composition according to claim 1 wherein the pharmaceutically acceptable carrier or excipient comprises a carbohydrate.

9. A method for the prevention or treatment of a disease or disorder selected from the group consisting of arthritis, rheumatoid arthritis, spondyloarthropathies, gouty arthritis, osteoarthritis, systemic lupus erythematosus, juvenile arthritis, asthma, hay fever, atopic dermatitis, rhinitis, bronchitis, COPD, cystic fibrosis, psoriasis, sclerodermia, morbus bechterew, sarcoidosis, tumor metastasis, morbus crohn, colitis ulcerosa, IBD, multiple sclerosis, arteriosclerosis, arteritis, myocardial infarction, stroke, coronary heart disease comprising the administration of an effective amount of a composition comprising a LTB₄ antagonist having a hydroxy and a benzamidine group or a tautomer, a pharmaceutically acceptable salt or solvate thereof (1) and a cyclooxygenase-2 or combined cox1/coxII inhibitor (2), to a patient in a combined form, or separately or sequentially.

10. The method according to claim 9 wherein the composition is administered to a patient for the prevention or treatment of rheumatoid arthritis, atopic dermatitis or coronary heart disease.

11. A method for the manufacture of a medicamentation for the prevention or treatment of disease or disorder selected from the group consisting of arthritis, including rheumatoid arthritis, spondyloarthropathies, gouty arthritis, osteoarthritis, systemic lupus erythematosus and juvenile arthritis, asthma, bronchitis, COPD and cystic fibrosis comprising mixing a LTB₄ antagonist having a hydroxy and a benzamidine group, or a tautomer, a pharmaceutically acceptable salt or solvate thereof (1) and a cyclooxygenase-2 inhibitor or combined cox1/2 inhibitor (2) in a combined form.

12. The method according to claim 11 wherein the medicamentation is effective for the prevention or treatment of rheumatoid arthritis, atopic dermatitis and coronary heart disease.

13. A pharmaceutical kit comprising at least two separate unit dosage forms (A) and (B) in which:
(A) comprises a composition containing LTB₄ antagonist having a hydroxy and a benzamidine group or a tautomer, a pharmaceutically acceptable salt or solvate thereof (1), and optionally a pharmaceutically acceptable carrier; and
(B) comprises a composition containing a cyclooxygenase-2 inhibitor or combined cox1/2 inhibitor, and optionally a pharmaceutically acceptable carrier or excipient.
